(19) United States
(12) Patent Application Publication
(10) Pub. No.: US 2016/0213917 A1
(43) Pub. Date: Jul. 28, 2016

(54) ENHANCED STABILIZING SYSTEM AND SURGICAL TOOL TO SECURE AN ELECTRODE ARRAY TO THE SPINAL CORE

(71) Applicant: UNIVERSITY OF IOWA RESEARCH FOUNDATION, Iowa City, IA (US)

(72) Inventors: Brian Dalm, Iowa City, IA (US); Steve Viljoen, Iowa City, IA (US); Matthew A. Howard, Iowa City, IA (US); Rob Shurig, Saint Paul, MN (US)

(21) Appl. No.: 14/916,892

(22) PCT Filed: Sep. 5, 2014
(86) PCT No.: PCT/US2014/054243

§ 371 Date: Mar. 4, 2016

Related U.S. Application Data
(60) Provisional application No. 61/874,340, filed on Sep. 5, 2013.

(51) Int. Cl. A61N 1/05; A61N 1/36

(52) U.S. Cl. CPC A61N 1/0558; A61N 1/36071; A61N 1/36067; A61N 1/36114

(57) ABSTRACT

Described in this disclosure is a support structure for an electrode array configured for implanting against the spinal cord for treatment of pain. The restoring force of the support structure works in concert with frictional coupling between the array and the spinal cord to overcome the inertial force associated with ateral motion of the array. The electrode array is supported with struts that run longitudinally across or near the array backing. For example, two struts can be affixed on either out-board longitudinal edge of the array. The electrode array can be further stabilized by securing to a vertebra in the patient using a strap equipped with a collar that encircles the bundle of electrical leads emerging from the array through the dura. Positional stability of the array is better than 0.5 nm per movement cycle, thereby inhibiting the array from lifting off of the spinal cord during movement of the spinal cord within the dura.

### REFERENCE TO RELATED APPLICATIONS

This application claims the priority benefit of U.S. provisional application 61/874,340, filed Sep. 5, 2013. The aforesaid priority application, along with the following published international applications, are hereby incorporated herein by reference in their entirety for all purposes: WO/2013/116368: Managing back pain by applying a high frequency electrical stimulus directly to the spinal cord; WO/2013/116377: System that secures an electrode array to the spinal cord for treating back pain; WO/2012/065125: Remotely controlled and/or laterally supported devices for direct spinal cord stimulation.

### FIELD OF THE INVENTION

The invention relates generally to the field of medical devices and pain management. In particular, it provides electrode arrays and support structures for electrical stimulation of the spinal cord.

### BACKGROUND

Chronic pain is an often unbearable sequelae of spinal cord injury or disease. It can interfere with the basic activities, effective rehabilitation, and quality of life of the patient. The prevalence of pain in patients with spinal cord injury is high: in some studies ranging from about 62% to 84% of patients. Back pain is also a feature of other injuries and conditions. For example, postural abnormalities and increased muscle tone in Parkinson's disease may cause back pain, where the prevalence can be as high as 74%. Other conditions associated with back pain include disc rupture, congestive heart failure and osteoarthritis.

Published international application WO/2012/065125 describes the construction and use of electrode arrays for stimulating the spinal cord. Subsequently published application WO/2013/116377 provides a technology whereby electrode arrays can be robustly secured after implantation, keeping the electrodes in contact with the spinal cord. Gentle pressure is maintained using a spring or support structure anchored to an anatomical feature or structure outside the spinal cord. Suitable anchor points include anatomical structures at the margins of the spinal canal (particularly the inner wall of the dura and immediately outside the spinal canal (exemplified by the vertebrae).

Because back pain is often intractable within the current spectrum of clinical modalities, new technology is needed for pain management.

### SUMMARY OF THE INVENTION

Described in this disclosure is a support structure for an electrode array configured for implanting against the spinal cord for treatment of pain. The restoring force of the support structure works in concert with frictional coupling between the array and the spinal cord to overcome the inertial force associated with ateral motion of the array. The electrode array is supported with struts that run longitudinally across or near the array backing.

One aspect of the invention is an implantable device for stimulating the spinal cord of a subject. Elements of the device include an array of electrodes arranged on a compliant backing that is configured to conform to the spinal cord, a deformable support structure configured to be secured to an anatomical structure inside or outside the dura and to urge the array towards the spinal cord, and one or more electrical leads or a lead bundle configured to pass from electrodes on the array through or against the support structure and out through the dura.

The support structure comprises one or more struts or rigid members positioned horizontally over or along the backing of the electrode array and configured to maintain disposition of the array on the spinal cord during movement of the spinal cord within the dura, thereby inhibiting the array from lifting off the spinal cord. For example, two struts can be affixed on or near the longitudinal edges of the array backing, configured to become oriented in a rostral caudal direction upon implantation of the array against a spinal cord. Such a device can be configured so that positional stability of the array when implanted against a spinal cord is better than about 0.5 nm per movement cycle, or can accommodate a total rostral-caudal motion of up to about 2 cm without lift-off of either end of the backing.

The device may also contain an attachment portion such as a strap that is configured for securing the device by way of one or more of its components (such as a lead or lead bundle) to a vertebra in the subject, for example, so as to bridge the lamina. The strap may be configured for securing secured to a vertebra of the subject at both ends of the strap (for example, to opposing lamina). The strap can be provided with a collar that is structured and arranged to conform to and grasp the lead bundle by at least partially encircling it. The collar is typically positioned on or near a long edge of the strap. Once the leads are secured in the collar, the implantable device is secured to the vertebra so as to maintain the array on the spinal cord during movement of the spinal cord within the dura. This inhibits the array from lifting off the spinal cord. The device may also have a clip configured to at least partially encircle the collar, thereby further securing the grasp of the lead bundle by the collar.

Alternatively or in addition to an attachment to a vertebrae, the device may contain an attachment portion configured for securing the connecting member(s) to a margin of the spinal cord dura, or to one or more dentate ligaments. The support structure may have one or more flexible loops between the array and the connecting member, positioned, for example, so as to urge the electrode array against the spinal cord, and may constitute or contain one or more electrical leads configured to supply electrical stimulation to the electrodes. There may also be a cuff portion configured to be joined with the dura at or near an access site during implantation of the device into the subject, thereby closing the access site.

Another aspect of the invention are various methods of implanting such a device onto the spinal cord of a subject. The device can be secured to a vertebra so that the electrode array is in contact with the spinal cord. This can be done by creating an incision in the dura over the dorsal aspect of the spinal canal of the subject, positioning the arrayed electrodes over the dorsal spinal cord at a location that is essentially symmetrical between the left and right dorsal root entry zones, lowering the support structure towards the spinal cord so as to compress a spring portion of the deformable support structure and engage the electrodes with the spinal cord within a desired pressure range, closing the incision around the connecting members, and securing the attachment portion to a vertebra of the subject. If the strap comprises a collar that is structured and arranged to conform to and grasp the lead bundle, the method includes engaging a bundle of electrical leads emanating from the device in the collar. The steps of the method can be performed in any order that achieves the intended objective, unless explicitly stated otherwise.

The implantable devices of this invention can be used for stimulating a spinal cord in a subject in need thereof, for example, to inhibit sensation of pain by the subject. Thus, this invention includes an implantable device as referred to above for use in treating back or leg pain, Parkinson's disease, spinal cord injury, or congestive heart failure.

Another aspect of the invention is surgical tool configured for securing and positioning an implantable device for implantation into a patient. The tool typically comprises a central arm, a coupling mounted on the arm and configured for securing the tool to a holding apparatus, upper and lower members, plates or surfaces projecting from the am that are substantially parallel and spaced apart by a defined span, a notch on an extremely of each of the upper and lower members (typically on the edge opposite the arm), the notch being configured to conform to a lead or lead bundle projecting from the implantable device, and an attachment on each of the upper and lower members, each configured to receive suture or other restraining means that binds the implantable device to the respective member.

The span can be defined such that when the implantable device is positioned beneath the lower member, the device may be secured to a strap passing through the span an as to bridge the lamina of a vertebrae of the patient such that the array of electrodes is urged against the patient's spinal cord. The attachment means on each of the upper and lower members can be a hole in the respective member between the extremely and the arm. There may be a plurality of holes arranged up the arm so that a suture securing the implantable device to the projecting members can pass through the holes, allowing the device to be released by severing the suture near the arm above the projecting members. The surgical tool can be provided in combination with an implantable device as outlined in the preceding paragraphs.

Another aspect of the invention is method of implanting an electrode array onto the spinal cord of a patient in need thereof using the afore-described tool. This can be done by securing the electrode array such as is already outlined to the surgical tool (for example, using suture), positioning the tool so that the electrode array contacts the spinal cord of the patient, surgically securing the electrode array to one or more anatomical structures of the patient (such as a vertebra), and then releasing the electrode array from the surgical tool. Accordingly, the invention includes a surgical tool as outlined above for use in treating back or leg pain, Parkinson's disease, spinal cord injury, or congestive heart failure.

Another aspect of the invention is a testing apparatus for assessing the stabilizing effect of a support structure for an electrode array. Such an apparatus may comprise a surrogate for a spinal cord, the surrogate comprising a tubular body with a rounded cross-section having a radius that approximates the radius of a human spinal cord, a holder configured to secure an implantable device for testing in the apparatus, wherein the implantable device comprises an electrode array joined with a support structure, wherein the holder is positioned such that when an implantable device is secured thereto, the support structure urges the electrode array against the surrogate, a staging system configured to adjustably position the surrogate relative to the holder to within a 1 mm tolerance in each of three dimensions, and a motor mechanically or operationally coupled to the surrogate such that activation of the motor generates cyclical lateral movement of the surrogate, thereby simulating rostral-caudal movement of a spinal cord within a thecal sac.

The testing apparatus can also include a control module configured and programmed to regulate the shaft speed to within 5% of its set point in hertz within a specified range. It can also include an inductive proximity sensor configured to monitor rotation of the motor's shaft, and a tachometer configured to count lateral motion cycles. The invention includes a method for determining the stabilizing defect of a support structure for an electrode array by securing an implantable device in the holder of the apparatus, moving the surrogate in a cyclical and lateral manner, and thereby assessing to what extent the support structure stabilizes the electrode array against the surrogate: particularly, the dynamic stability and lift-off of the electrode array from the surrogate.

Further aspects of the invention appear in the description that follows and the appended claims.

### DRAWINGS

FIGS. 1A to 1D provide an end view, aside view, a perspective view, and a top-down view of an array device having struts in accordance with this invention. The device comprises an electrode bearing portion 11, a spring portion 12 to urge the electrodes against the spinal cord; a dural cuff 14; a connecting member 15; and a bundle of electrical leads 16. The lead bundle can be secured to the vertebrae using a titanium strap.

FIGS. 2A to 2F show the design and use of a securing strap with a collar. FIG. 2A is an upper perspective view of a collar 42 designed for securing onto one edge of the strap 41 using screws. FIGS. 2B and 2C show alternative designs with the collar integrated with the strap. FIGS. 2D and 2F show two alternative clip designs, both of which partly encircle the collar, thereby increasing the grip of the collar on the lead bundle to secure the bindle more firmly. FIG. 2E is a side view of the collar-clip arrangement.

FIGS. 3A to 3E are views of a surgical tool for use in implantation of an electrode array device. FIG. 3A shows an anterior lateral view with a central support arm 50 with two projecting members 51a and 51b on the head 51, where the electrode array device is secured during surgery. FIGS. 3B and 3C provides details of the head 51. FIG. 3D shows a superior view of the tool highlighting the notch 52a at the end of the upper projecting member 51a for acceptance of the electrode array device; and holes at the end of the head 54a and 54b for passage of suture used to secure the electrode array device to the tool. FIG. 3E shows a superior lateral view of the top portion of the tool, with the coupling 57 for securing to a holding apparatus.

FIGS. 4A to 4E illustrate a general procedure for implanting an electrode array device into a patient using the surgical tool. A standard thoracic laminectomy is performed (FIG. 4A), and the electrode array device is secured to the tool (FIG. 4B). The electrode array device is lowered on spinal cord, and the table mounted apparatus is locked in position to provide rigid fixation (FIG. 4C). The rostral portion of the dural cuff 14 is sutured to the native dura (FIG. 4D). The suture securing the apparatus to the tool is cut so the tool can be removed, with a metal strap 41 securing the lead bundle 16 of the device to the lamina 26.

FIGS. 5A and 5B show an electrode array with support structures to stabilize the array during movement of the spinal cord. The array is shown with and without struts affixed onto the axial out-board edges of the support structure.

FIG. 6 is a conceptual physical arrangement for an electrode support structure. A lateral view of the spinal cord is shown surrounded by CSF inside the dura.

FIG. 7A shows a prototype of an electrode array and its support structure in full view. FIG. 7B shows a close-up of the electrode array on the underside showing the struts.

FIG. 8A is a computer-aided design image showing a profile view of the test rig, with each of the major sub-assemblies identified. FIG. 8B is a front-view image of the fully assembled system.

FIG. 9 shows an electrode array functionally coupled to a prototype support structure having struts. It is shown mounted on an apparatus configured to test the response to movement of a spinal cord. The electrode-bearing surface is in contact with the dorsal aspect of a spinal cord surrogate.

### DETAILED DESCRIPTION

This disclosure provides improved structural features for an electrode array device designed for implantation directly in contact with the spinal cord for purposes of electrical stimulation and pain relief. As a result of these features, the array resists lift-off from the spinal cord, avoiding the possible adverse consequences of inadequate contact of the electrodes during patient movement, or repositioning of the array away from the target treatment site.

As described in more detail below, the electrode array can be equipped with stabilization struts that add stiffness in the longitudinal direction of the electrode array, while allowing the patch to retain flexibility in the circumferential direction of the spinal cord. As the spinal cord moves in either the rostral or caudal direction, the stiffness provided by the struts restricts the patch from bending excessively which allows the electrode array residing in the patch to retain intimate contact with the surface of the spinal cord. Positional stability of the array can be better than 0.5 nm per movement cycle. The electrode array can be further stabilized by securing to a vertebra in the patient using a strap equipped with a collar that encircles the bundle of electrical leads emerging from the array through the dura.

This disclosure also provides a surgical tool designed to secure and position an electrode array during implantation in a patient to be in contact with the spinal cord.

### Implantable Electrode Array Device

The general structure of an implantable device of this invention comprises several components, as described and illustrated in WO/2012/065125. First, an array of electrodes is arranged on a compliant backing that is configured to conform to the spinal cord. Upon implantation in the spinal canal, the electrodes face towards and contact the dorsal surface of the spinal cord, with the backing supporting them and keeping them in the desired pattern or configuration in contact with the spinal cord. Typically, the backing is substantially rectangular, configured so that the longer side is parallel to the spinal cord in a caudal-rostral orientation.

An implantable device typically comprises a plurality of electrodes for placing in direct contact or electrical communication with the pail surface and underlying white matter of the spinal cord, within the spinal canal. The electrodes may be arrayed on a pliable background, constructed of a material and in a shape that allows it to be conformed directly to the spinal cord. The electrodes may be supplied with stimulating power through a common lead. Alternatively, the electrodes may be attached singly or in groups to separate leads so that each electrode or electrode group can provide the spinal cord with a separate stimulus as programmed by a central control unit.

The device further comprises one or more electrical leads operatively connected on one end to one or more of the electrodes, and on the other end to a power source that is programmed to provide a charge to all or some of the electrodes in the array in a programmed pattern that stimulates the spinal cord in a way that achieves the desired clinical effect. The connection with the power source may be wireless, or the support structure and the leads may be configured so that the leads pass from electrodes on the array through or against the support structure and out through the dura.

### Electrode Array Device Having a Support Structure

The device can be provided with a deformable support structure configured to be secured to an anatomical structure inside or outside the dura and to urge the array towards the spinal cord: in particular, to one or more dentate ligaments, the inside surface of the dura, one or more vertebrae, or any combination thereof. This is described and illustrated in WO/2013/116377.

For securing to a vertebra, the device comprises an electrode array with a spring portion or support structure that maintains contact of the electrodes in the array with the spinal cord. The spring portion or element is configured (by a choice of shape, thickness, rigidity, and distance away from the array itself) to exert a pressure by the array on the spinal cord within a predetermined or desired range upon implantation of the device into a subject. The range of this pressure can be 0.1 mm Hg through 25 mm Hg. Various mechanical spring shapes are suitable for this compliant element. Exemplary is one or more flexible loops that are attached to the upper structure of the device on one side and to the array on another side. For economy of design and operation, the spring portion may consist of or contain a portion of the electrical leads supplying stimulation to the electrodes.

The support structure may integrally comprise an attachment portion for securing directly to a vertebra. Alternatively, it may comprise one or more vertical connecting members configured for securing to a separate strap that bridges the lamina. The strap may have any suitable shape that spans between and secures to the lamina or other parts of the vertebra within a suitably confined volume The strap may be packaged or provided together with other components of the device in kit form, or supplied separately.

Other possible components include a dural cuff portion attached to the vertical connecting member and configured to be joined with the dura at or near an access site during implantation of the device into the subject, thereby closing the spinal canal. A scaffold portion may be attached to the vertical portion between the cuff portion and the spring portion, configured so as to be positioned beneath the access site after closure. There may also be an electrical connector at or near the position where the device exits the dura, whereby electrical leads passing from the electrodes through the vertical member(s) to the connector may be electrically and reversibly connected to a power source.

### Electrode Array Device Having Struts

This invention provides an implantable device in which the array is supported by one or more struts, rigid members or a framework positioned above, alongside, or along or near the dorsal side of the backing opposite from the outer or lower surface from which the electrodes project. As further described below, the struts, members or framework is configured to maintain disposition of the array on the spinal cord during movement of the spinal cord within the dura, thereby inhibiting the array from lifting off the spinal cord, with the result that the electrodes would no longer be in contact with the spinal cord.

The term “strut” as used in this disclosure refers to a structural piece designed to resist pressure and transverse flexing in the direction of its length. It can be in the form of a singular or isolated rod or bar, or combined with other features thereby forming part of a framework. The rigid member is a physiologically compatible material in a typically linear form that is less pliable than the electrode backing, although it is sufficiently pliable so that it conforms to and moves with the spinal cord. Struts may be affixed directly to the electrode array backing or directly to the array support structure, or both in any effective orientation.

Typically, upon implantation into the subject, one, two, or more than two struts are oriented so as to be substantially parallel with the longer side of the electrode backing or with the spinal cord, with a strut located above, on, or near the long side of the electrode backing. A suitable framework could comprise one or more transverse members connecting the parallel struts to each other in a manner that helps maintain the general orientation of rostral-caudal oriented struts. The transverse members may be thicker, stronger or more pliable than the backing but less so than the struts that run parallel to the long side of the backing,

An electrode support structure having struts works in concert with frictional coupling between the array and the spinal cord to overcome the inertial force associated with lateral motion of the array. Some of the array support structures of this invention comprise struts that are positioned over or on the side of the array. Positional stability of the array is preferably better than 0.2, 0.5 or 1 nm per movement cycle. A spinal cord stimulator device preferably accommodates a total rostral-caudal motion of at least 0.5, 1, or 2 cm of the cord/membrane relative to the fixation point, i.e., at least 1 cm rostral and 1 cm caudal from the neutral position.

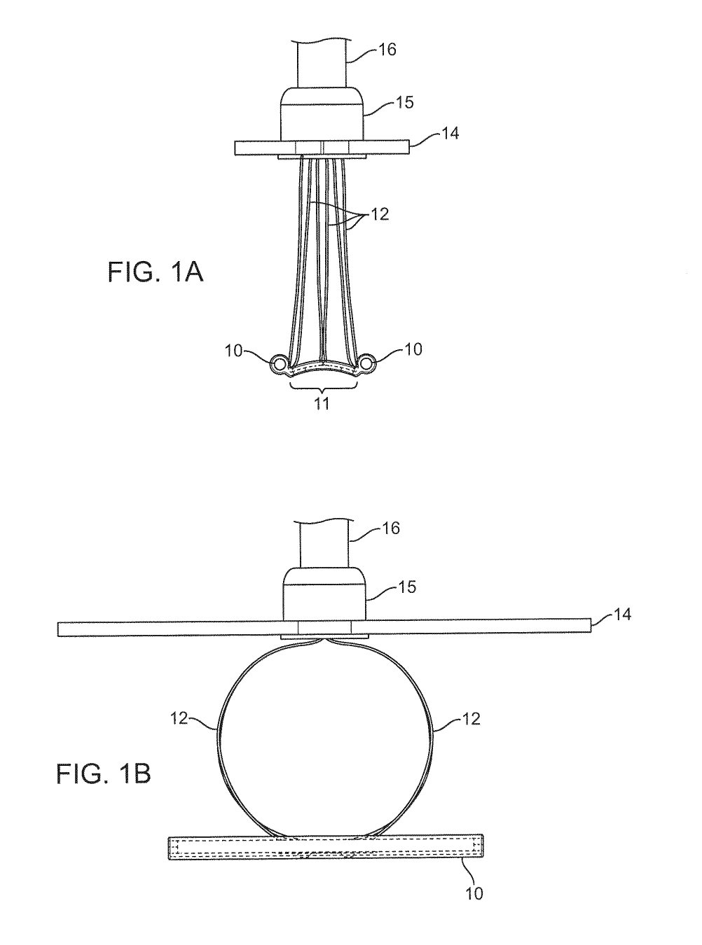

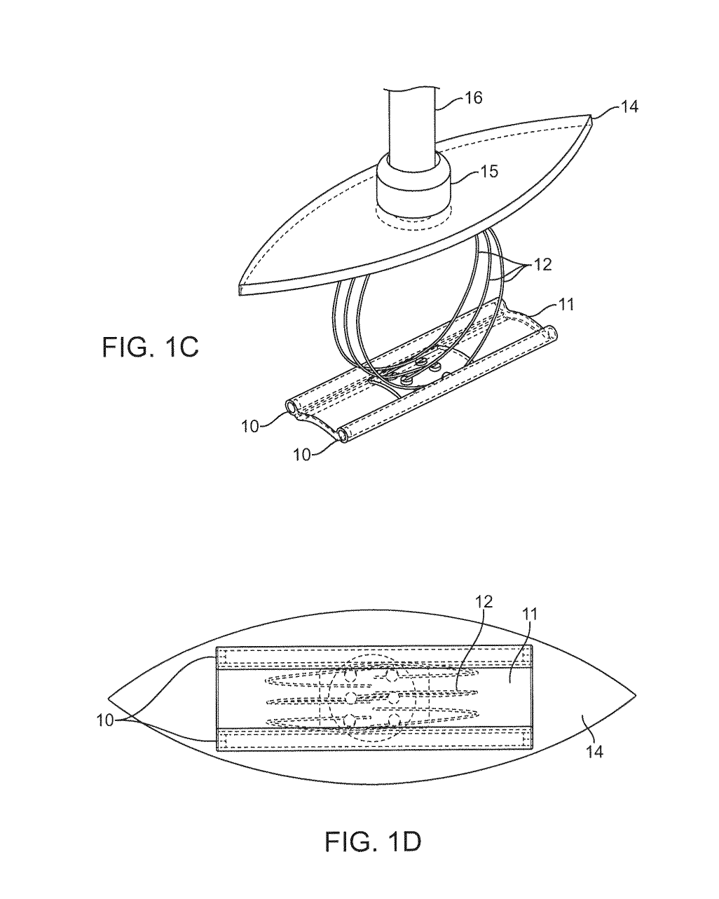

FIGS. 1A to 1D provide different views of an array device having struts. The device comprises an electrode bearing patch or portion 11 with electrodes projecting from the under surface for direct contact with the spinal cord, The electrode bearing patch 11 is configured to contact and conform to the spinal cord, and has a deformable spring portion or support structure configured to urge the electrodes of the array into contact with the spinal cord during movement of the spinal cord within the spinal canal. In this illustration, the spring portion is fashioned the shape of a plurality of loops 12, Also shown is a dural cuff 14 that is situated and sized to be sutured or glued in continuity with the dura, a dome-shaped connecting member 15 that connects to a bundle of electrical leads 16 passing upwards from the support structure 12. The struts 10 add beneficial functional features to the electrode bearing portion 11 by providing rigidity or resilience (in combination with the spring portion) to help keep the electrodes in place on the spinal cord during movement of the spinal cord within the backbone.

The support rods may be dark in color (a shade of a color that is at least 50% and optionally at least 70% or 90% black). The dark color contrasts with the light coloring of the spinal cord, and allows the stabilization struts to be used as markers for location and orientation of the electrode array patch.

### Electrode Array Device Having a Clamping System

The electrode array device can be anchored to a vertebra in the patient by securing a strap to a vertebra of the subject during implantation. Typically, the strap is configured to be attached to opposing sides of the vertebra (particularly, to the lamina) at each end of the strap. This invention provides the strap with a retention cuff or collar that facilitates surgical procedures and has certain functional benefits.

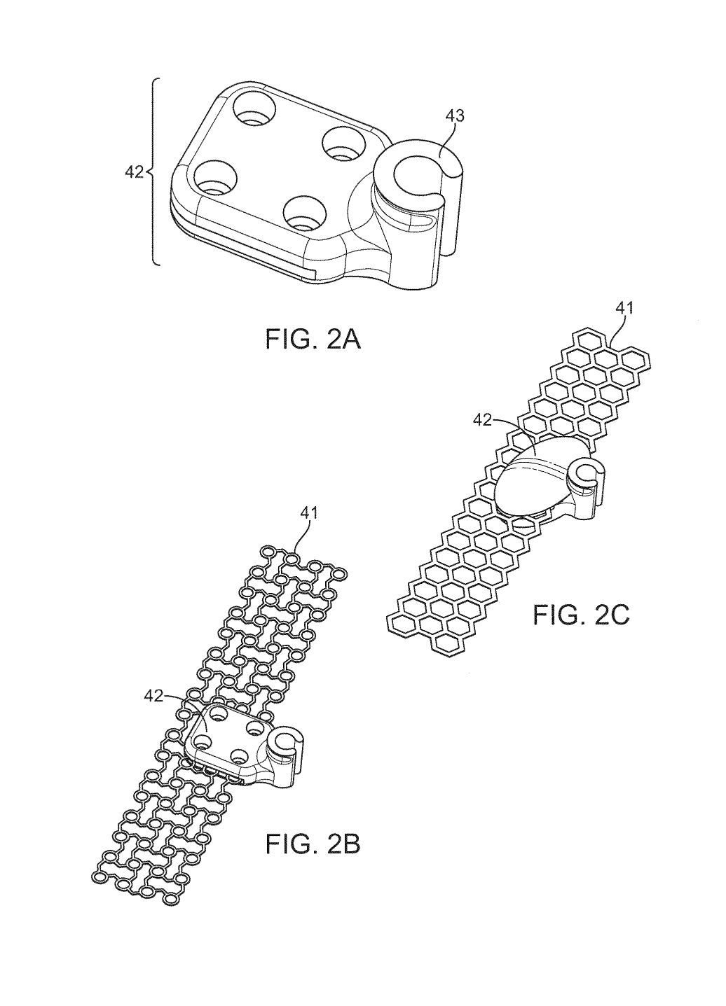

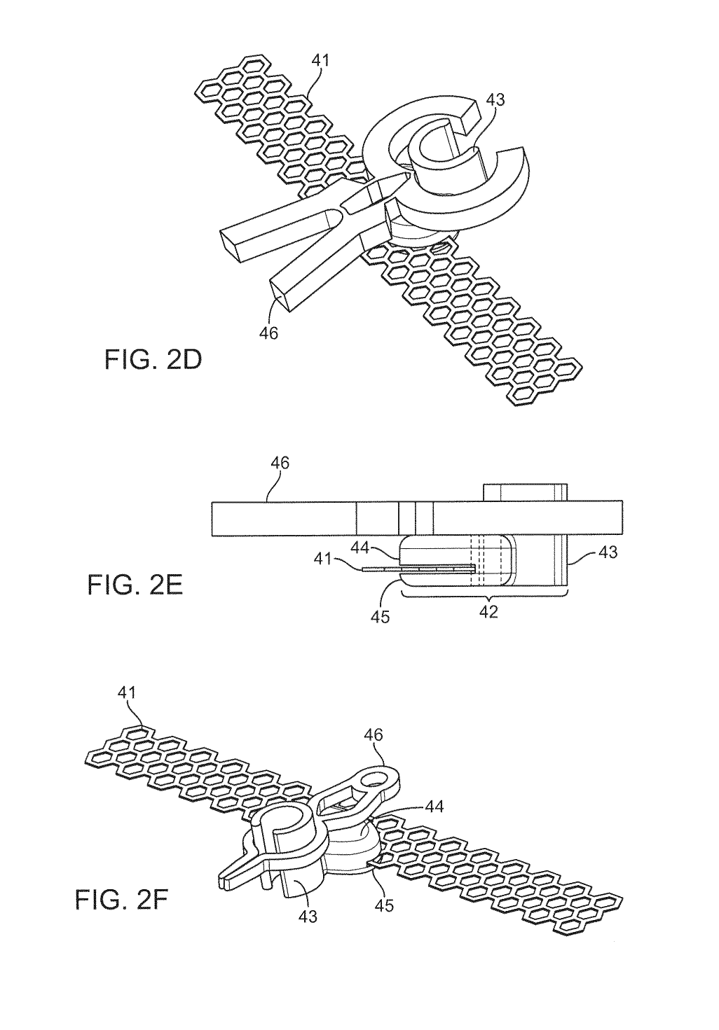

FIGS. 2A to 2F show the design arid use of a securing strap with a collar. FIG. 2A is an upper perspective view of a collar 42 designed for securing onto one edge of the strap 41 using screws. FIG. 2B shows the collar 42 integrated with the strap 41 at about midway along the long edge on one side. The length of the strap here is about 3.5 inches. FIG. 2C shows an alternative design where the collar 42 is fused or bonded to the strap 41 without the use of screws. The length of the strap here is about 4 cm. The strap is made of a surgically compatible resilient material, typically a light metal with high breaking strength such as titanium. The strap shown here is constructed in the form of a mesh to reduce weight and facilitate bending. The collar is made of any suitable thermoplastic, organic, or silicon material that is sufficiently pliable to be inserted about the lead bundle, but resilient enough to provide a permanent grip.

The inner diameter of the collar 42 is sized to grasp the lead bundle 16 emanating from the electrode array just outside the dural closure, accommodating any sheath or covering that may surround the bundle. Use of the collar on the strap is integrated with the surgical procedure as follows. First, the titanium mesh is bent to a desired shape to bridge between attachment points on the vertebra, and engage the electrode array device in the middle. The collar grips onto the lead bundle of the electrode array device by pushing it over the lead bundle. The titanium mesh is anchored to the vertebra, typically but not necessarily after the collar has engaged the bundle.

To increase the grip of the collar on the lead bundle, the collar can be provided with a groove around its circumference in which the surgeon may tie one or more loops of suture. Alternatively, one or more retention clips can be used. FIGS. 2D and 2F show two alternative clip designs, both of which partly encircle the collar, thereby increasing the grip of the collar on the lead bundle to secure the bindle more firmly. The clip mechanism may have a small snap feature to lock it in the closed position. Silicone pads adhered to the distal portion of the clip in the inside diameter can be used to compliment the silicone cuff to hold the lead bundle.

FIG. 2E is a side view of the collar-clip arrangement showing the collar 42 with its bundle-encircling portion 43 attached to an upper 44 and lower 45 portion that surround and are bonded to the strap 41. The inner diameter of the clip 46 completely or partly encircles the outer diameter of the collar 42, thereby increasing the clamping pressure on the lead bundle.

In FIG. 2F, a fenestrated aneurysm clip is used, having an inside diameter of 5 mm, and a force of about 150 grams. Two or more separate clips can be used in combination to hold the lead bundle with adequate firmness. Titanium aneurysm clips suitable for this purpose are Mizuho™ 006-40, and Aesculap™ FT637T or FE637K. In general terms, the clamping or closing pressure of the collar and the clip(s) in combination is at least 25 grams, preferably at least 100, 200, or 300 grams, or in the range of 25 to 500 or 100 to 300 grams.

### Apparatus for Positioning the Electrode Support Device During Installation

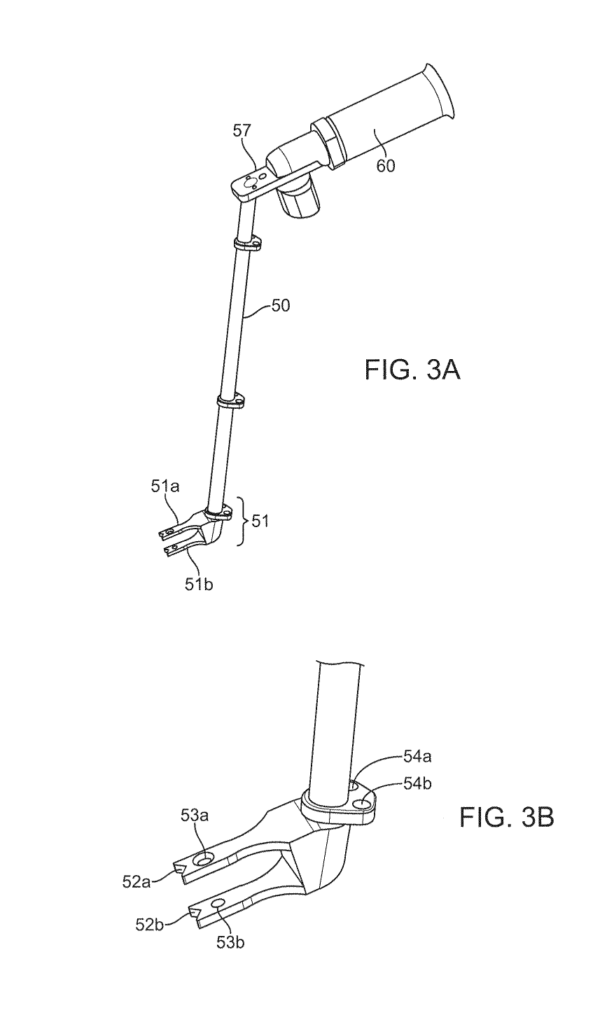

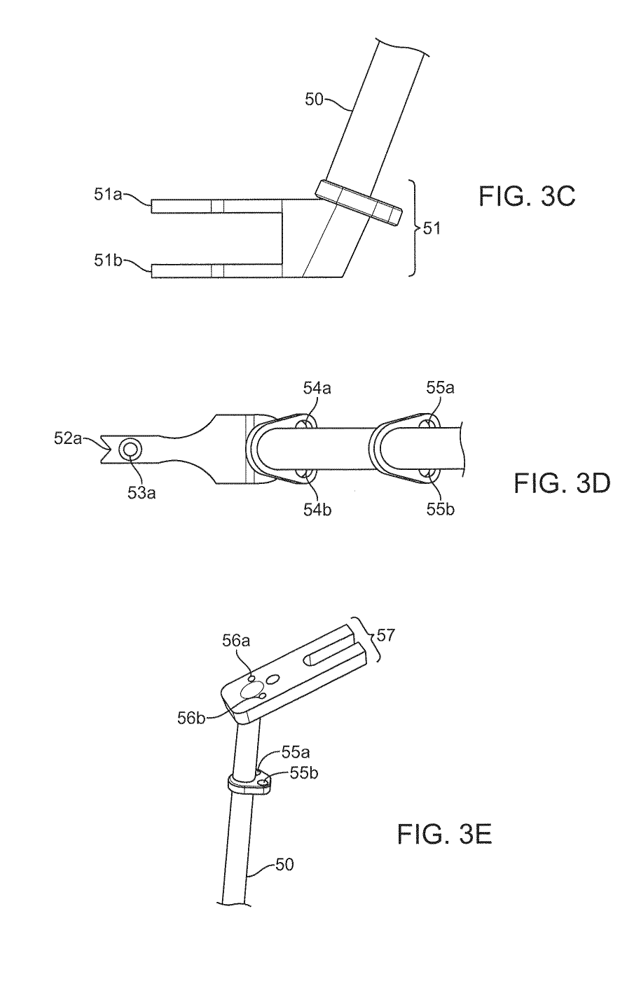

Another aspect of this invention is a surgical tool that is designed and configured to hold an array device securely in position during implantation into a patient. The device positioning apparatus (DPA) is illustrated and exemplified in this disclosure by the Iowa Patch Applier™ (IPA), as depicted in FIGS. 3A to 3E.

The surgical tool has a coupling that is configured for attaching to and being secured by a holding apparatus, such as a table mount, retractor, or stand, a floor stand or support, or a surgical robot. The coupling means can be any joint that reversibly couples to and secures an insertion or donor member on the surgical tool to a complementary receiving member on the holding apparatus. By way of example, such a joint could comprise a clamp arrangement, a rod coupling, a socket-plug arrangement, or their equivalents.

The coupling means is attached on the tool to a base or backbone structure, referred to in this disclosure as an arm. The term includes any substantially rigid structure that connects the coupling referred to in the last paragraph with the head of the tool, described in the following paragraph. It may have any shape or structure, and is optionally adjustable in length and/or confirmation. When in use, the arm tends to be positioned downwards (substantially vertical or at a downwards angle), depending on the nature and location of the holding apparatus in relation to the operating workspace.

The head of the tool (typically the lowermost portion when in use comprises two substantially horizontal and substantially parallel plates or members projecting from the arm. They are configured such that an array securing system being implanted in a spinal cord can be secured underneath the lower member. At the center of the outermost projecting edge of both members is a concave hollow or notch that conforms to the upper portion of the electrode array device above the cuff, comprising the bundle of electrical leads. The array device can be micasably secured to the surgical tool by using a suture to encircle or pass around the outside of the lead bundle such that the bundle is engaged by and secured in the notch of the upper and lower projecting members, drawing the device by way of the lead bundle along each projecting member back towards the arm.

To assist in this arrangement, there are typically one or more suture securing or attachment means on each projecting member, located between the notch and the arm, that the suture can be drawn to or through, such as a hole, loop, post, or clamp. Using a hole or loop allows the surgeon to pass the suture through the securing means, back to the arm for tying there, or through one or more other holes or loops providing a pathway back up the arm, optionally to a tying position at or near the top of the arm where the arm couples to the holding apparatus. Amongst the advantages of this arrangement is that the surgeon can conveniently release the array device from the tool by cutting the suture anywhere up the pathway.

The two projecting members on the head provide two contact points that prevent sagittal jostling of the array device. The projecting members may be constructed to be narrow, allowing for adequate line of site for the surgeon.

The distance between the two parallel projecting members is chosen in reference to the sizing of the array device and the anatomical distances in the subject where the device is to be implanted. The array device is secured to the tool such that the cuff used to seal the dura after placement abuts or is adjacent to the lower surface of the lower of the two projecting members. After the dura is closed, a metal strap can be used to secure the array device to the backbone by passing it through the spacing between the projecting members, where it engages or is secured to the lead bundle of the device, and then screwing the strap to the backbone on either side. With this in view, spacing between the lower surfaced of the upper member and the upper surface of the lower member is typically chosen to be about 0.4 to 1.4 cm, 0.5 to 1.0 cm, 0.4 to 0.7 cm, or 0.6 to 0.8 cm.

FIG. 3A shows an anterior lateral view of a prototype IPA with a central support arm 50. It has a head 51 with two projecting members 51a and 51b, where the electrode array device is secured. At the top is a coupling 57 where the IPA is engaged by a holding apparatus 60 that keeps the electrode array device in position during implantation.

FIG. 3B highlights the notches 52a and 52b at the end of the two projecting members 51a and 51b of the head piece that serve as attachment points to accept the electrode array device. There is also a hole 53a on the top projecting member 51a and a hole 53b in the bottom projecting member 51b that allow suture to pass through to secure the electrode array device to the IPA. The base portion of the arm 50 had two eyelets 54a and 54b through which the suture can pass upwards.

FIG. 3C shows a lateral enlarged view of the head 51 of the IPA. It highlights the distance between the two projecting members of the head 51a and 51b which serves as a space to allow for the metal strap attachment.

FIG. 3D provides a superior view of the IPA highlighting the notch 52a at the end of the head for acceptance of the electrode array device; holes at the end of the head 54a and 54b for passage of suture used to secure the electrode array device to the IPA. The head portion in this example is narrow along the length compared to the base to facilitate a direct line of sight visualization of the array device as it is lowered into the precise location on the spinal cord surface. Eyelets 55a and 55b further up the arm 50 are configured so that that suture passes through on its way to the top portion of the IPA where it can be tied and secured.

FIG. 3E shows a superior lateral view of the top portion of the IPA, with the coupling 57 for securing to a holding apparatus 60. This view highlights the eyelets 55a and 55b along the top portion of the arm, and two holes 56a and 56b at the top to allow passage of the suture where it will ultimately be secured.

### Process for Securing the Device to the Vertebrae

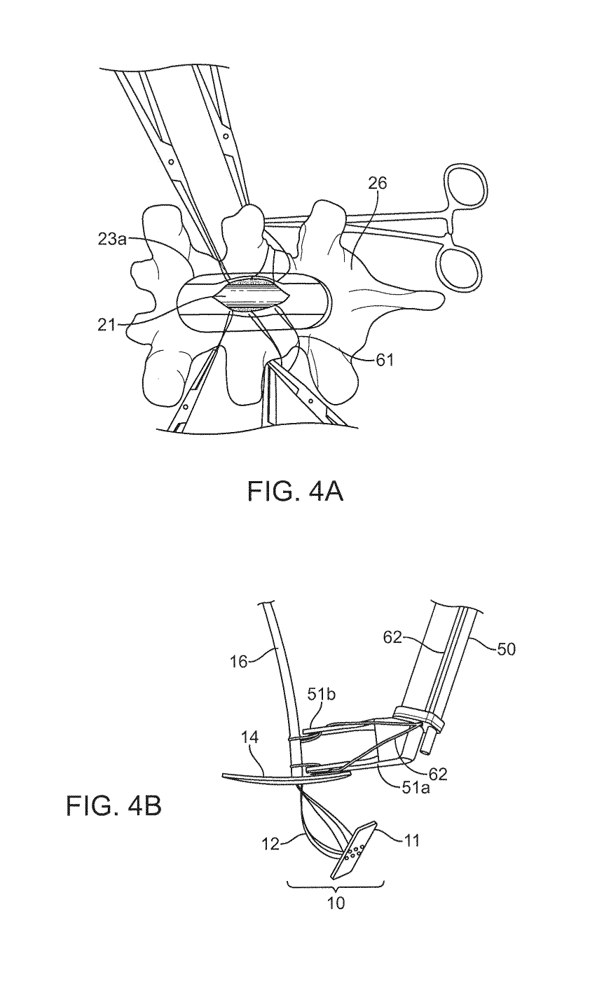

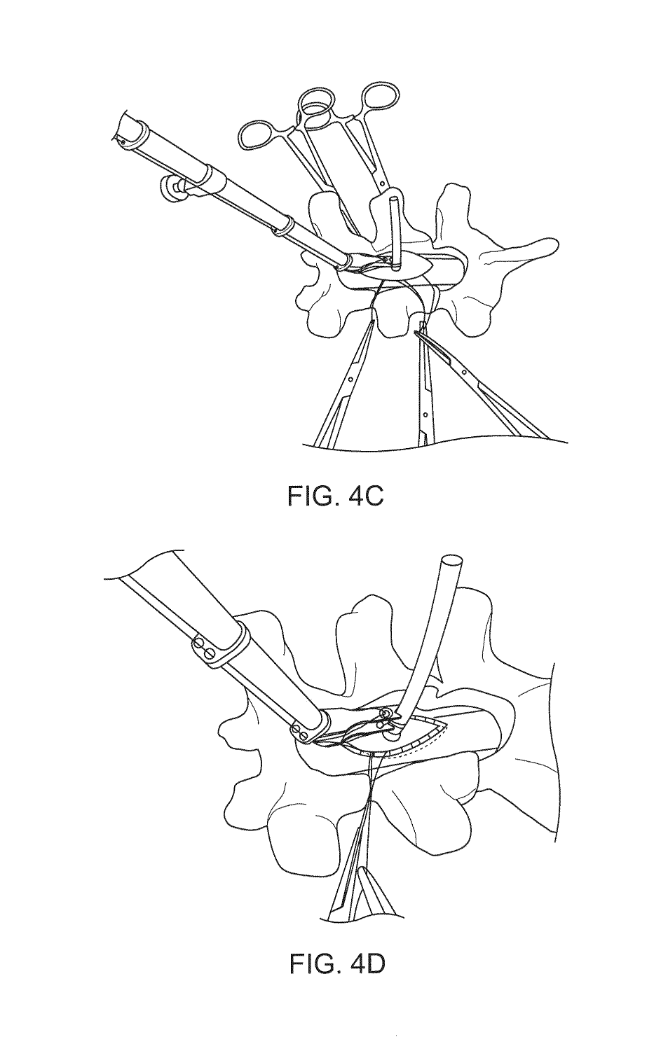

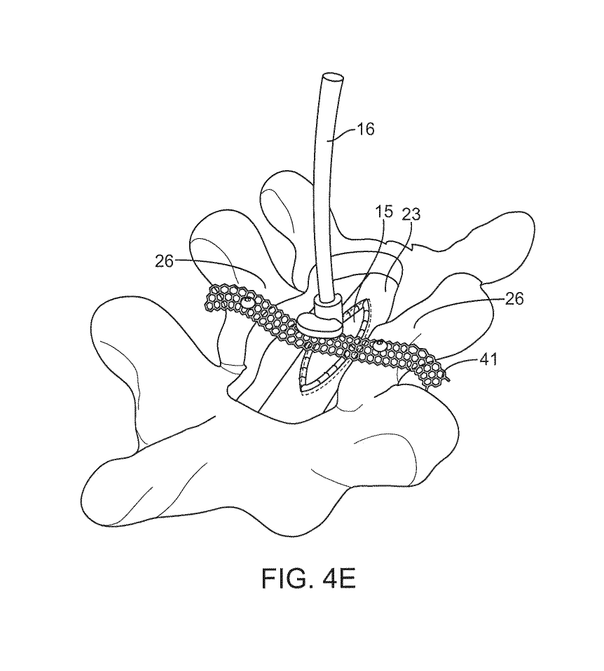

FIGS. 4A to 4E are perspective views providing illustrations in which an electrode array according to this invention may be secured to a vertebra of the subject .

In general terms, the surgeon should have clear line-of-sight visualization of the top of the electrode bearing portion of the device as it is positioned on the surface of the spinal cord. The distance between the electrode bearing portion of the device and the point at which the leads fuse into the dural cuff exit site can be set precisely during the operation. This sets the tension of the malleable intradural leads at the optimal force under conditions when the spinal cord is at its most ventrally displaced position within the spinal canal.

With reference to FIGS. 4A to 4E, placement of the electrode array device can generally be done in accordance with the following steps:
Step 1. (FIG. 4A), A standard thoracic laminectomy is performed in the vertebrae 26 on a patient in need of treatment, and a midline dural opening 23a is made approximately 3 cm in length. The dural edges are retracted with suture 61 exposing the spinal cord 21.
Step 2. (FIG. 4B). The electrode array device 10 intended for implantation in the patient is secured to the projections 51a and 51b of the IPA surgical tool using suture 62. Upon implantation of the electrode array device into a patient, the electrode bearing portion 11 will be placed on and conform to the surface of the spinal cord, and kept in place by way of a spring portion 12 that presses the array against the cord. The suture is passed around the extradural portion of the electrode array device and is then brought through the holes in the head of the IPA and along holes adjacent to the arm and secured at the top portion of the IPA.
Step 3. (FIG. 4C). The IPA with the electrode array device is lowered onto the spinal cord and placed under direct visualization in the optimal position with the use of a table mounted apparatus to which it has been coupled. Once positioned, the table mounted apparatus is locked in position to provide rigid fixation of the IPA.
Step 4. (FIG. 4D), The rostral portion of the dural cuff is then sutured to the native dura and the rostral ⅔ of the dural closure is performed.
Step 5. The titanium strap is then placed over the latninectomy defect. The cuff of the titanium strap is placed precisely between the two heads of the IPA. The strap is secured to the laminae with bone screws.
Step 6. A suture is placed around the titanium strap cuff to secure the extradural portion of the electrode array device to the titanium strap.
Step 7. The suture securing the electrode array device to the IPA is cut, allowing a smooth release.
Step 8. The IPA is removed from the field. As shown in FIG. 4E, this leaves the electrode array device implanted in position with the dural cuff 15 sutured to the dura 23, and the lead bundle 16 extending, upwards. The metal strap 41 secures the lead bundle 16 and is screwed to the lamina 26.
Step 9. A standard thirty-degree endoscope is introduced through the caudal dural defect and proper placement of the electrode array device is verified prior to final dural closure.
Step 10. The caudal dura is closed.
Step 11. The remainder of the procedure including generator placement and relay lead tunneling proceeds in a standard fashion.
Step 12. The wound is closed.

Thus, the electrode bundle 16 is secured by way of the securing strap 41 to the vertebra 26. This maintains pressure of the electrode array 11 to the spinal cord 21 within a desirable range of pressure. The electrodes maintain a position where they stimulate the spinal cord without losing contact should the spinal cord move from its neutral position, avoiding injury to the spinal cord and surrounding tissues, and not provoking an inflammatory response.

Depending on the nature of the device, the anatomical dimensions and physiological requirements of the subject, and the judgment of the surgeon, a suitable pressure could be anywhere from 0.1 mm Hg to 25 mm Hg. The upper limit (25 mm Hg) is about half of a typical low range human diastolic blood pressure, so that blood flow through surface vessels on the spinal cord would not be choked off by the pressure applied to them by the electrode-bearing surface.

### Device Components and Commercial Distribution

A device or assembly according to this invention may be part of a system that also comprises any external components: particularly a power supply, and a control unit that sends control signals to the circuitry or electrodes on the implanted device. Typically, the external source will provide electronics for controlling the electrical stimuli. There may be a microprocessor or other suitable controller that is programmed to shape the electrical stimuli into one or more particular patterns, and to regulate the frequency of an alternating current. The external component of the device may be configured to receive operator input regarding stimulus pattern selection and/or amplitude and frequency, Alternatively or in addition, the external component may also be configured to receive feedback data and to adjust the pattern and/or amplitude and frequency to improve the effect perceived by the patent.

Optionally, the circuitry controlling the stimulus supplied by the electrodes may be built into the same backing as the electrodes. Power and control signals can be provided to the circuitry and the electrodes by electrical leads that pass in and out through the dura. Alternatively, the device may have a receiving means such as an antenna through which to receive power and control signals wirelessly from an external source.

For some purposes, the device may be supplied from the manufacturer in a standard size that can accommodate almost the full range of spinal cord anatomy variations encountered in patients. Alternatively, the device can be built in a plurality of different standard sizes with struts of different lengths, or may be custom manufactured for particular patients. In these circumstances, the method of installing the device would further comprise the step of determining appropriate dimensions of the patient's anatomy (such as circumference or cross-sectional shape of the spinal cord and/or the spinal canal on the dorsal side, and/or dimensions of the vertebra to which the device is to be secured.

### Clinical Use

The device and technology of this invention can be used for diagnostic, therapeutic, and research purposes in human subjects, primates, and other domesticated and non-domesticated mammals. Upon determination that a human patient or other subject would benefit from electrical stimulation from a device according to the invention, the clinician would first implant the device onto the spinal cord. The location may be predetermined by imaging the spine and/or doing neurological studies, and then selecting a location that would be most likely to convey the desired benefit.

The device is implanted by conforming the arrayed electrodes to a region of the spinal cord so that the electrodes directly contact the spinal cord, and then securing the device in place. Once fixed in place, it remains after surgical closure, and maintains the electrodes in contact with the spinal cord, notwithstanding normal pulsation and mobility of the spinal cord, movement of the patient in ordinary daily activity, and movements resulting mechanical such as might result if the patient slips or falls. The affixing of the device, while robust, is preferably reversible so that the device can later be removed or repositioned if:needed, while causing minimal damage to the tissues.

Once implanted, the electrode array can be used for stimulating a spinal cord of a patient. The patient may be subject or susceptible to noxious or deleterious nerve signals transmitted along the spinal cord, or otherwise requires treatment. An electrical stimulus is provided through the electrodes in the array directly to the spinal cord so as to inhibit transmission of such noxious or deleterious nerve signals.

The stimulus may be applied to inhibit sensation of pain, or to inhibit symptoms or sensory input that is undesirable or disruptive to the patient, either in the back itself, the extremities, or at another location wherein the pain is mediated at least in part by the spinal cord. Conditions suitable for treatment include back pain, leg pain, Parkinson's disease, spinal cord injury, Failed Back Surgery Syndrome, arthritic degeneration, phantom limb pain, numbness or palsy, or congestive heart failure. The stimulus may be provided to the spinal cord by the device on a constitutive basis, in response to feedback data, or it may be subject to the patient's conscious control.

The treating clinician may select any electrical stimulus that is effective in managing pain of a particular patient. The general object is to induce refractoriness of the spinal cord to transmit noxious or deleterious signals or synchronous depolarization events initiated locally. This can be adjusted empirically by determining neural activity and recording the symptoms experienced by the patient.

WO/2013/116368 describes different patterns of stimulation that may be effective in accordance with clinical circumstances. One option is to provide an electrical stimulus with a pattern having a sufficiently high frequency to inhibit sensory side effects such as paresthesia (numbness or tingling). Under control of an appropriately programmed microprocessor or any other suitable type of controlled signal generator, electrodes in the array may all provide the same signal pattern, or individual or groups of electrodes may have their own signal pattern configured to work independently or in concert with signal patterns of other electrodes in the array.

Depending on the objective of the treatment and the manner in which the technology is deployed, effective pulse repetition rates or frequencies may be a 1,000 Hz, 4,000 Hz, or 10,000 Hz, or a frequency range of about 1,000 to 9,000 Hz. The electrical potential may vary at a regular frequency in a sinusoidal or square wave form. Alternatively, the wave form may be a more complex pattern, with pulses appearing at varying intervals and intensities according to a calculated or repetitive pattern. Such patterns comprise a pulse train generating substantially continuous activation of nerves within the spinal cord, and may incorporate irregular pulse intervals, irregular pulse amplitudes, a variety of wave forms, or any combination thereof. The potential may create what is essentially abroad band noise, varying at stochastic or essentially random intervals and intensity under the influence of a suitable computational algorithm or automated control program in a microprocessor.

The benefit of this approach may be attributed to the fact that bundles of sensory axons fire randomly when not transmitting sensory stimulus. When a sensory stimulus is presented, a substantial proportion of the axons within a bundle or pathway will discharge in a synchronous fashion—firing axons potentials at about the same time. This results in the sensory input being transmitted along the axons in the bundle, so that the subject may experience the sensation.

Patients with leg and back pain may have bundles of axons spontaneously firing in a synchronous manner, instead of the normal random pattern of firing. A low frequency alternating current (50 Hz) may be effective in reducing the sensation of pain, but the stimulation may generate neurological side effects such as paresthesias (an effect that is sometimes experienced as tingling or numbness). However, a high frequency electrical stimulus (say, about 5,000 Hz) has interval spacing shorter than the refractory period of axons. By delivering electrical pulses at high frequency, the relative timing of tiring by individual axons within the bundle of axons becomes nearly random, with different axons becoming excitable again at different times. Applying high frequency pulses to the spinal cord can be used to restore a state of active quiescence in the sensory nerves passing through the cord.

Treating back pain may comprise administering an effective electronic stimulus to the spinal cord, monitoring transmission of synchronous action potential through the spinal cord or inferring the same, and then adjusting the electrical stimulus an as to further inhibit transmission through the spinal cord of synchronous action potentials.

The electrical stimulus may be adjusted in frequency or other waveform parameters and manner of application so as to minimize side effects such as paresthesia, and to minimize impact on transmission of essential neurological faction, including motor neuron activity, and nerves involved in proprioception and kinesthesia. Optionally, the clinician or the user may be provided with an input or control means to select the pattern, adjust the frequency, and adjust the intensity in accordance with the perceived symptoms.

### EXAMPLES

### Example 1

### Measurement of Spinal Cord Motion During Flexion of the Spine in Human Subjects

For purposes of this study, a 1.5 T Magnetom Espree® magnet (Siemens, Erlangen, Germany) was used. Informed consent was obtained from healthy volunteers ranging in age from 23 to 58. Each volunteer was first imaged in a supine neutral position and then imaged in a maximal attainable flexed position.

To obtain the maximal flexion of the spine, patients were given three basic positioning instructions. The first was to rotate their pelvis backwards towards the gantry as far as possible to remove the lumbar lordosis and straighten the lumbar spine. The second was to curl their upper back, neck and head forward so that their shoulders were as close to their knees as possible. The third instruction was then to tuck their chin down as close to their chest as possible. While attaining this flexed position in the bore, a variety of foam wedges and pillows were used for added support so that the patient could remain as still as possible during image acquisition.

Imaging was obtained in the coronal plane. Three-dimensional multiplanar reconstruction software was used on a Carestream PACS station to aid in measurement, The T10 and T11 nerve roots were identified. A cranial caudal measurement was made in a plane parallel to the spinal canal between the dorsal-root entry zones (DREZ) of T 10 and T11. (The exact: position of the entry zones was confirmed by assessing sequential axial images to identify the most cranial aspect of the nerve originating from the spinal cord) the difference between this measurement on the neutral and flexed images is a measure of spinal cord contraction/expansion along the rostral-caudal axis. Next, a cranial caudal measurement was made from the DREZ of the T10 nerve root along the same plane as the prior measurement, to the level of a plane orthogonal to the spinal canal at the level of the inferior T10 pedicles. The latter were selected as a reference point of the bony canal inside of which the spinal cord moves. The difference between these measurements represents cord movement within the bony canal.

Results were as follows. The spinal cord should move rostrally during flexion and should lie in its most caudal location when the patient is in the neutral position. The measured change in the pedicle-to-spinal cord DREZ distance across all patients between the neutral and flexion positions ranged from 1.9 mm to 18.0 mm, with a mean and standard deviation of 8.5±6.0 mm. The inter-DREZ distance across all patients between the neutral and flexion positions ranged from −2.0 mm to +6.7 mm, with a mean and standard deviation of 3.5±2.6 mm. The mean and standard deviation for the rostral-caudal conus movement was found to be 6.4±4.1 mm within an overall range of 1.1 to 11.4 mm. The fractional variations in these findings (standard deviation mean) are very large, 71%, 74% and 64% respectively. This reflects the wide variability in the capacity of individual subjects to maximally flex the spine, as well as possible inter-subject variability in spinal cord mechanical characteristics. These findings highlight the need for the device to accommodate larger patient-to-patient variations in spinal cord dynamic movement properties.

The ratio of the spinal cord's mean stretch-to-mean axial movement over a full flexion cycle was 3.5 mm/8.5 mm ≈40%. On average across all patients, it required 1 mm of net axial displacement of the cord to stretch it 0.4 mm in length. A spinal cord stimulator device preferably accommodates a total rostral-caudal motion of at least 1 cm of the cord/membrane relative to the fixation point i.e., 1 cm rostral and I cm caudal from the neutral position.

Since there were large variations (70%) in the magnitude of that motion from patient to patient, there will be a spectrum of spinal cord strains associated with flexion-driven motion of the cord. Having suitable axial compliance within the electrode bearing portion of the device will reduce the risk of potential irritation of the pial surface in patients where the intraparenchymal strains are large. In patients with small levels of strain, there would be little relative motion between cord and the array, meaning that there would be small risk of any skidding between them. The net axial travel of the spinal cord relative to the fixation point is within the range that can be accommodated without lift-off of the electrode bearing portion of the device.

### Example 2

### Loop Support Structure With Struts

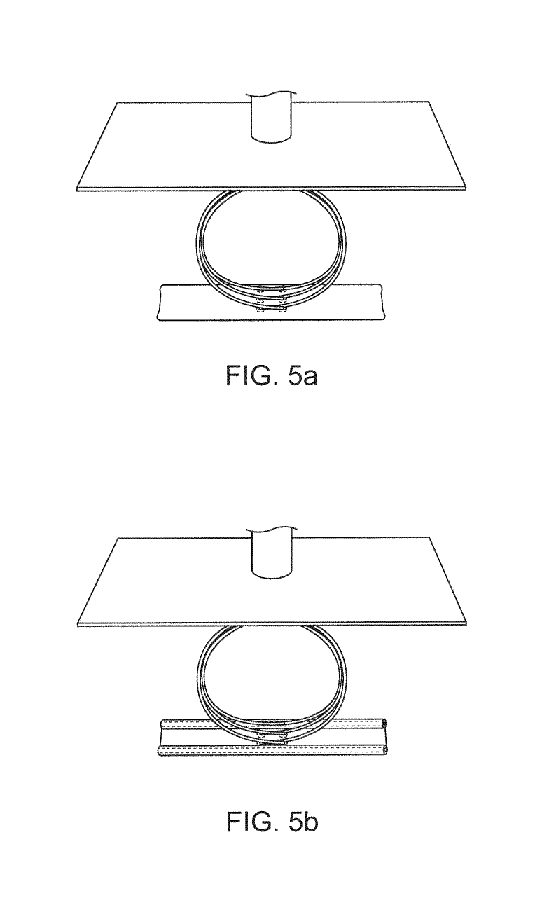

FIGS. 5A and 5B are close-up images of a 12 mm prototype electrode array and array support device with and without support struts on the electrode-bearing surface.

The support struts are introduced into the array support structure for the purpose increasing the flexural stiffness of the electrode-bearing surface, in order to maximize the travel range before its axial tips lifted off the spinal cord surrogate. The prototypes shown in FIG. 5B were made of polyetheretherketone (PEEK) tubing, approximately 0.5 mm in diameter and 15 mm long. In the clinical version, the support struts would instead be of a stiff grade of silicone and be incorporated into the structure of the electrode-bearing surface itself.

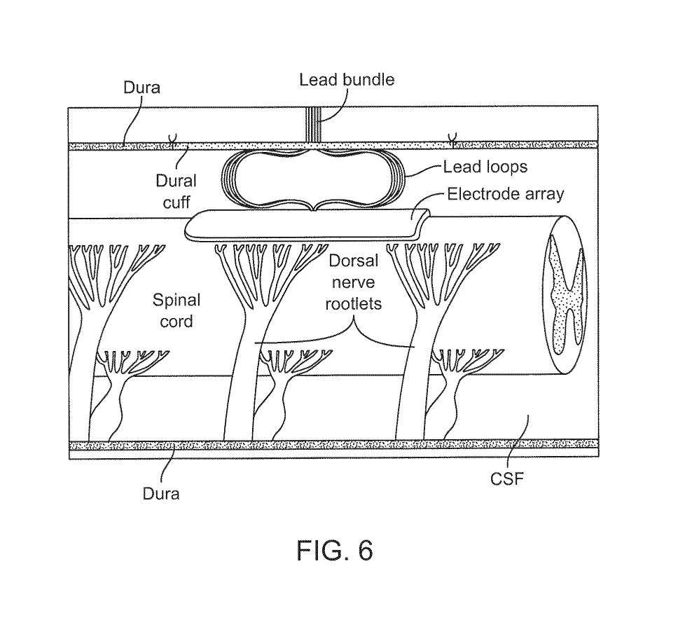

FIG. 6 shows a conceptual physical arrangement for an electrode support structure. A lateral view of the spinal cord is shown surrounded by CSF inside the dura. The electrode-bearing implant rests on the dorsal (back-facing) surface of the spinal cord. It is kept gently in place by the soft compliance (ranging from 2 to 60 μN μm⁻¹) of the lead loops of the electrodes. The leads exit the thecal sac through the durotomy opening, which is then sealed by a bioresorbable dural cuff, as is the standard practice for durotomy. Fixation of the entire assembly to the encasing vertebra is by a titanium bridge that spans the laminectomy. The lead bundle is subsequently connected to an implantable pulse generator which produces the stimulus signals that the electrodes apply to the spinal cord tissues.

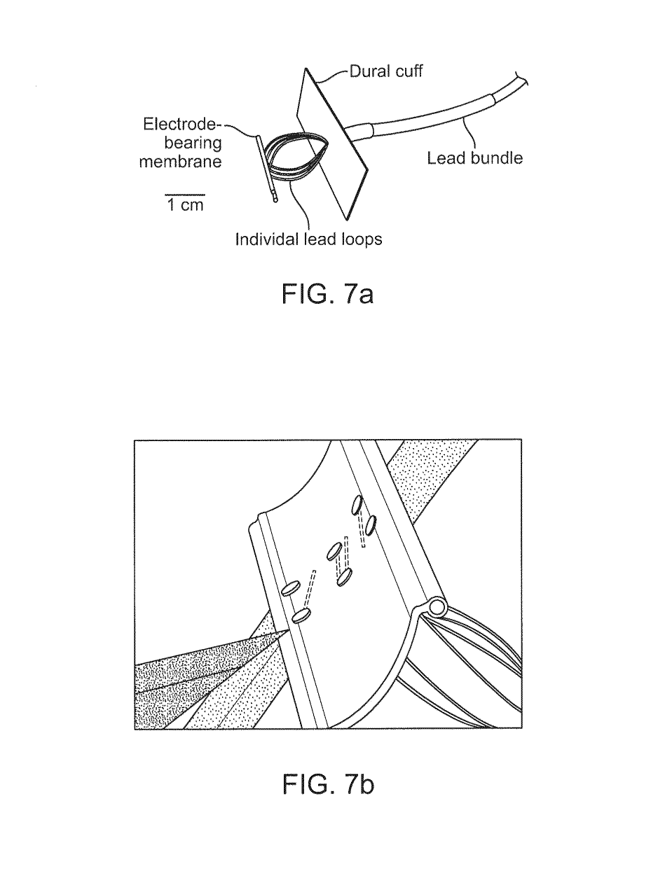

FIG. 7A shows a prototype of an electrode array and its support structure in full view. FIG. 7B shows a close-up of the electrode array on the underside, with the struts running parallel to the array on each side. The implant is made of biocompatible silicone, approximately 16 mm long, 6 mm wide and 1 mm thick, and has been formed with an ˜4 mm radius of curvature to accommodate the roughly oval shape of the thoracic-level spinal cord. The leads are of 0.1 mm diameter, insulated, 35N LT® Ag-core stranded cables, and the electrodes are flat platinum disks, 0.7 mm in diameter. S. Viljoen et al., J, Med, Biol. Eng. 33, 193 (2013).

### Example 3

### Apparatus for Simulating Dynamic Interactions Between the Spinal Cord and Intradural Implants

The invention provides bench-top instrumentation that mimics important aspects of electrode array implantation on the mobile spinal cord. The instrumentation comprises a reciprocating motion control system that employs an anthropomorphic surrogate of the human spinal cord for use in range-of-travel measurements of the array.

The apparatus simulates the reciprocating large amplitude movements of the spinal cord with adjustable speed and range of motion, makes the moving element in that apparatus an anthropomorphic surrogate of adult human spinal cord, and incorporates a fixture for mounting prototype electrode support devices on the surrogate spinal cord with adjustability of the degree of compression of the lead loops and their angle of approach to the electrode-bearing surface.

### Design Requirements for Motion Testing Apparatus

Except when tethered because of a disease process or from scar formation, the spinal cord is virtually in continuous motion inside the spinal canal. Even in the otherwise quiescent patient, there are at the very minimum the vascular and CST-driven pulsations of the cord, which can produce low amplitude (<0.5 mm) oscillatory motions at speeds up to 7 mm/sec. M. A. Howard et al., J. Appl. Phys. 110, 044702 (2011).

Biophysical considerations guided the design process for the testing apparatus. First, in healthy adults, trunk flexions of up to 120° can be achieved over approximately 1 s, hence the nominal stroke rate for reciprocating movements of the spinal cord surrogate should be on the order of 1 Hz, with the ability to operate at faster speeds in order to accelerate testing cycles, increase strain rates, etc. Second, although the spinal cord follows the curve of the spinal canal over its full length, its sub-centimeter range of motion during flexion is small enough to allow approximation by a flat surrogate rather than a curved one. Third, the surrogate spinal cord should have an elastic modulus that closely matches that of actual spinal cord, for accurate simulation of the interaction dynamics between the electrode array and the living tissues. Lastly, because the human body spends roughly as much time oriented vertically as horizontally, the apparatus should be capable in principle of operating in any orientation, in order to simulate various postural positions.

### System Build-Out

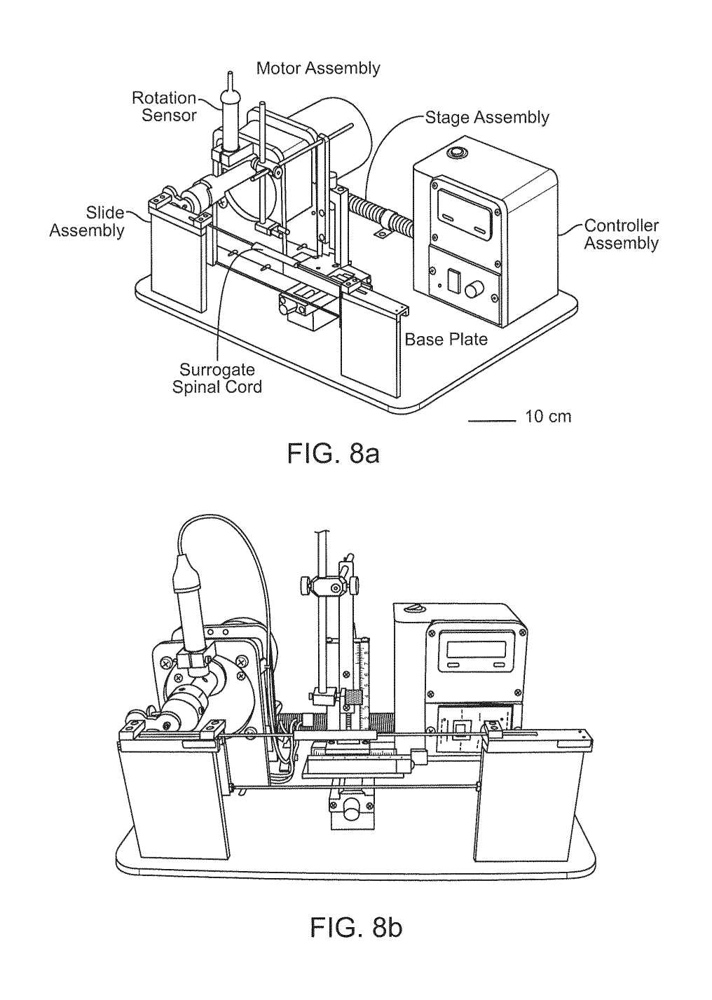

FIGS. 8A and 8B show the fully assembled test rig in conceptual profile and in full-view, respectively. There are two central features of the apparatus. The first is its ability for precise placement of the electrode array electrode array at any chosen location on the upper surface of the surrogate spinal cord, which is described in detail in the next subsection. This is enabled by the stage assembly. The second is that it can reliably carry out large numbers (>10⁷) of repetitions of specific, user-defined spinal cord displacement cycles. The motor, controller and slide assemblies serve that purpose.

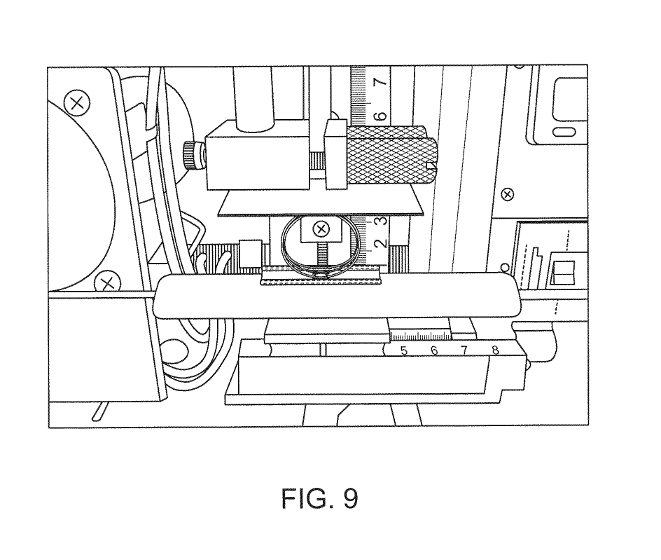

FIG. 9 is a close-up showing the lead bundle exiting the top of the dural cuff of the electrode array is fixed inside a clamp at the bottom of a riser rod which provides coarse vertical adjustment for the positioning of the electrode array on the surrogate spinal cord. The riser rod is coupled to a lateral stand-off rod by a post clamp, with the stand-off rod subsequently fixed to a Velmex A15 Series translation stage which provides fine vertical adjustment along the Z axis (1 mm of stage travel per turn of the lead screw). Two additional such translation stages provide for similarly precise X-and Y-axis adjustment of the electrode array location.

Cyclical lateral motion of the surrogate is used to simulate the rostral-caudal movement of the spinal cord within the thecal sac. A pair of linear ball slides (Parker Hannifin, model 3507-20) serve as opposing mounting points for a thin stainless steel tube which passes axially through the cord surrogate, thus establishing the lateral translation pathway. The linear slides are also inter-connected by a rigid stainless steel rod in parallel with the tubelsurrogate assembly, in order to provide the axial stiffness needed to insure that the slides move in unison. A double-joint end linkage (Igus Inc., model EGZM1-04-25, 25 mm between bearing centers) couples the in-board linear slide to an adjustable boring head (Sherline Products Inc., model 3049) mounted on the motor shaft. The boring head serves as the cam in this arrangement. The mounting point on it to which the linkage is attached can be offset from the motor's axis of rotation, thus providing a lever arm of up to 22 mm with a resolution of 0.02 mm. This enables adjustment of the stroke length of the linear slide to a precision of approximately 0.1%.

The motor and gear box (SPG Co., models S9140GEH-V12 and S9DB6B1HA respectively) are driven by a control module (SPG Co., model SUA401A-V I 2A) capable of regulating the shaft speed to within 5% of its set point over the range from 15 to 283 rpm (0.25-4.7 Hz). An inductive proximity sensor (Red Lion Controls, model PSAC0000) monitors the rotation of the motor's shall and signals a digital tachometer (Red Lion Controls, model CUB5R000) that can totalize up to 10⁸ counts, i.e., lateral motion cycles. All components of the apparatus are mounted onto a 0.6×30×40 cm anodized aluminum base plate for stability. Because everything is fixed in place securely, the apparatus could be used in any orientation, but is typically run with the base plate resting on a flat surface.

### Spinal Cord Surrogate

FIG. 9 also shows a spinal cord surrogate used in testing. This is described in detail in M. A. Howard et al., J. Appl. Phys. 110, 044702 (2011). It is constructed of silicone with an oval cross section of 6 mm minor diameter and 10 mm major diameter, and with a length of 6 cm. The silicone formulation used for it has an elastic modulus in the range of 0.41 to 0.44 MPa, which matches well with that derived from low-strain rate measurements made on ex vivo samples of human spinal cord.

A central support rod passes through it axially, to serve as the actuator shaft for the cyclic lateral motions. It consists of a 30 cm length of 16 gauge, 304 stainless steel hypodermic needle tubing, approximately 1.6 mm in outer diameter. It is incorporated into the surrogate by passing it through carefully aligned center holes in the end-caps of the mold into which the silicone mix is poured. After a 12 hour cure, the mold components are separated and the surrogate with integral support rod.

### Example 4

### Testing motion Control in Electrode Arrays Supported by Struts

The mechanical performance characteristics of electrode support devices with three different lead-loop diameters, 12 mm, 10 mm, and 8 mm were investigated in the testing apparatus described in the previous example.

### Testing Protocols

Testing was carried out with the testing apparatus described in the previous example. The testing comprised (1) evaluations of the dynamic stability of the electrode array on the spinal cord surrogate as the latter was driven in lateral reciprocating motion, and (2) static determinations of the extremes of motion at which the tip of the electrode array began to lift off the surrogate.

Both types of test were done as a function of the compression depth of the lead loops, in order to mimic the naturally occurring changes in dura-spinal cord distance associated with postural shifts and movement of the spine. The dynamic motion studies were typically at a drive rate of 2 Hz, i.e., two complete translational movement cycles of the spinal cord surrogate per second, thus allowing for the accumulation, e.g., of 10⁶ cycles in just under six days of run time. Video recordings of brief segments (˜10 s each) of the motion sequences were made at various intervals in order to enable visual inspections of the electrode array behavior during the translation cycles. All observations and measurements were archived for subsequent assessment.

Each device was evaluated in terms of how the electrode-bearing surface coupled to the spinal cord surrogate over many different combinations of loop compression and lateral displacement amplitude. The testing apparatus was used to test the effects of loop diameter on the positional stabilities of the prototypes, and also to see if there were conditions under which the loops became entangled when subjected to physiological-scale compressions and cord movements.

### Dynamic Stability

After positioning the electrode array in the stage assembly, reference lines were drawn on the spinal cord surrogate to mark the initial locations of the leading and trailing edges of the electrode-bearing surface. Motor-driven translational movement of the surrogate was then initiated and carried out until at least 300,000 cycles, and typically twice that many, had been achieved. Visual and video-monitor observations of the electrode array location relative to the reference lines during testing revealed that for all three sizes of device, every combination of movement amplitude (within the lift-off limits, see below) and lead-loop compression led to maintenance of the initial position of the device on the spinal cord surrogate over the entire test. The edge of each reference line could be resolved visually to within roughly 0.2 mm. Over 500,000 cycles of testing, the slippage or positional drift of the electrode array on the spinal cord surrogate would have been less than 0.4 nm per translational cycle.

The dynamic effects that lead to this level of stability were investigated. For a stroke length of 1 cm occurring at 2 Hz, the maximum displacement of 5 mm from the neutral position would be achieved in 0.125 s. The mean speed over that period would be 5 min/0.125 s=40 mm/sec=0.04 m/sec, and thus the mean acceleration would be 0.32 m s⁻². The electrode-bearing surface of the electrode array has a mass of about 0.13 grams=1.3×10⁴ kg; hence the mean lateral inertial force acting on it during that part of the movement cycle is 0.42 μN.

The force exerted by the lead loops on the electrode-bearing surface of the electrode array was measured as function of loop diameter and compression depth. The 12 mm device exhibited a compliance of approximately 2.4 4 μm⁻¹. For a typical compression depth of 5 mm, the resulting force was 11 mN which is >10^{∝}times the orthogonal inertial force estimated above.

A significant finding of the stability tests was in regard to the electrical patency of the electrode array leads. Thin strips of metallic film were placed between the bottom of the electrode array and the dorsal surface of the surrogate in such a way as to allow interconnection of the electrodes and leads in series, so that the overall input-to-output conductivity of the device could be monitored by ohmmeter during testing. In one study with the 10 mm device, in which the range of motion was ±5.25 mm at 2 Hz, the leads were still patent at 1.3 million cycles with no indications of impending loss of robustness.

### B. Lift-off Measurements

The testing apparatus was also used for static determination of the extremes of motion at which the distal tip of the electrode array began to lift off the surrogate. This is a performance parameter for the device because repeated lift-off from and re-contact with the spinal cord by the distal tips of the device would likely lead to injury of the pial surface.

Table 1 summarizes the results of the measurements which were done by simply moving the surrogate manually forwards and backwards relative to the center-line of the suspension point until 0.5 mm of lift-off was observed.

| | Maximum axial displacement with ≦0.5 mm lift at distal | | | | | |
|---|---|---|---|---|---|---|
| | edge of the electrode bearing surface (positive axial | | | | | |
| | displacement/negative axial displacement, mm) | | | | | |
| | 12 mm Loop | | 10 mm Loop | | 8 mm Loop | |
| | Without | With | Without | With | Without | With |
| Dural cuff to | support | support | support | support | support | support |
| cord distance | struts | struts | struts | struts | struts | struts |
| 15 mm | 0 | 1/6 | | | | |
| 14 mm | 1/0 | 3/7 | | | | |
| 13 mm | 0/2 | 5/7 | | 1/1 | | |
| 12 mm | 2/3 | 7/7 | 3/1 | 4/4 | | |
| 11 mm | 1/2 | 6/7 | 1/1 | 4/6 | | |
| 10 mm | —/1 | 8/7 | 3/2 | 5/7 | 1/0 | |
| 9 mm | — | 10/7 | 2/2 | 7/8 | 3/2 | 0/1 |
| 8 mm | — | 9/7 | 2/4 | 9/8 | 4/2 | 2/4 |
| 7 mm | —/1 | 9/11 | 3/3 | 10/8 | 5/3 | 3/6 |
| 6 mm | 1/4 | 9/10 | 1/1 | 10/9 | 4/3 | 5/6 |
| 5 mm | 3/4 | 9/14 | 1/1 | 9/10 | 5/1 | 6/7 |
| 4 mm | 2/4 | 7/9 | 1/1 | 11/10 | 6/4 | 7/8 |
| 3 mm | | | 2/1 | 10/7 | 5/2 | 7/9 |
| 2 mm | | | | | 4/2 | |

The data were taken as a function of device type and degree of loop compression, and with and without support struts along the axial edges of the electrode bearing surface. The results show that the support struts extend the range of travel significantly for the 10 and 12 mm devices as lead loops are compressed, with the findings being more equivocal for the 8 mm device.

The testing apparatus was also used to investigate one of the variables associated with the surgical procedure. In closing the durotomy after implantation of the electrode array, it is likely that the vertical centerline along the diameter of the lead loops will not intersect the axial midline of the spinal cord, but instead will be offset laterally by perhaps as much as ±5 mm matter how carefully the dura is dissected. By adjustment of the horizontal position of the electrode array suspension point using the translation stage, the effects of such offsets on the range of travel before lift-off the distal tips of the device could be investigated.

The results of tests carried out on all three devices at the full 5 mm of lateral offset are presented in Table 11.

| | Maximum axial displacement with ≦0.5 mm lift at distal | | | | | |
|---|---|---|---|---|---|---|
| | edge of the electrode bearing surface (positive axial | | | | | |
| | displacement/negative axial displacement, mm) | | | | | |
| | 12 mm Loop | | 10 mm Loop | | 8 mm Loop | |
| | +5 mm | −5 mm | +5 mm | −5 mm | +5 mm | −5 mm |
| Dural cuff to | lateral | lateral | lateral | lateral | lateral | lateral |
| cord distance | displacement | displacement | displacement | displacement | displacement | displacement |
| 15 mm | | | | | | |
| 14 mm | | | | | | |
| 13 mm | 5/3 | | | | | |
| 12 mm | 6/7 | 6/7 | | | | |
| 11 mm | 7/8 | 7/8 | | | | |
| 10 mm | 8/10 | 8/8 | 3/4 | | | |
| 9 mm | 9/9 | 7/8 | 3/4 | 5/3 | | |
| 8 mm | 7/8 | 8/9 | 6/6 | 6/6 | | |
| 7 mm | 8/10 | 8/8 | 6/7 | 8/6 | | |
| 6 mm | 9/10 | 9/9 | 7/8 | 7/8 | 4/4 | 2/4 |
| 5 mm | 10/10 | 10/9 | 8/9 | 8/9 | 4/4 | 3/5 |
| 4 mm | 11/11 | 5/11 | 7/9 | 8/7 | 6/5 | 5/7 |
| 3 mm | | | 7/8 | 7/9 | 5/3 | 7/5 |
| 2 mm | | | | | | |

The data show symmetry of response to positive vs. negative offsets, except at the maximum value of compression for the 12 mm device.

Each and every publication and patent document cited in this disclosure is hereby incorporated herein by reference in its entirety for all purposes to the same extent as if each such publication or document was specifically and individually indicated to be incorporated herein by reference.

While the invention has been described with reference to the specific embodiments, changes can be made and equivalents can be substituted to adapt to a particular context or intended use, thereby achieving benefits of the invention without departing from the scope of what is claimed.

The invention claimed is:

1. An implantable device for stimulating the spinal cord of a subject, comprising:
(a) an array of electrodes arranged on a compliant backing that is configured to conform to the spinal cord;
(b) a deformable support structure configured to be secured to an anatomical structure inside or outside the dura and to urge the array towards the spinal cord; and
(c) one or more electrical leads configured to pass from electrodes on the array through or against the support structure and out through the dura;
wherein the support structure comprises one or more struts or rigid members positioned horizontally over or along the backing of the electrode array and configured to maintain disposition of the array on the spinal cord during movement of the spinal cord within the dura, thereby inhibiting the array from lifting off the spinal cord.

2. The implantable device of claim 1, comprising two struts affixed on or near the longitudinal edges of the array backing, configured so as to be oriented in a rostral caudal direction upon implantation of the array against a spinal cord.

3. The device of either preceding claim, which is configured so that positional stability of the array when implanted against a spinal cord is better than 0.5 mm per movement cycle

4. The device of any preceding claim which accommodates a total rostral-caudal motion of 2 cm without lift-off of either end of the backing

5. The device of any preceding claim, further comprising an attachment portion configured for securing the support structure to a vertebra in the subject,

6. The device of claim 5, wherein the attachment portion comprises or is configured for securing to a strap, said strap being secured to lamina of a vertebra of a subject so as to bridge the lamina.

7. An implantable device for stimulating the spinal cord of a subject, comprising:
(a) an array of electrodes arranged on a compliant hacking that is configured to conform to the spinal cord;
(b) a deformable support structure configured to urge the array towards the spinal cord;
(c) a bundle of electrical leads configured to pass from electrodes on the a through or against the support structure and out through the dura; and
(d) a strap configured to he secured to a vertebra of the subject at both ends of the strap;
wherein the strap comprises a collar mounted on or near a long edge of the strap, the collar being structured and arranged to conform to and grasp the lead bundle so as to secure the implantable device to the vertebra and maintain disposition of the array on the spinal cord during movement of the spinal cord within the dura, thereby inhibiting the array from lifting off the spinal cord.

8. The device of claim 7, further comprising one or more clips configured to at least partially encircle the collar, thereby increasing closing pressure of the collar on the lead bundle.

9. The device of claim 7 or claim 8, wherein the strap is configured to bridge and be secured to opposing lamina of the vertebra.

10. An implantable device according to any of claims 1 to 6, further comprising the strap, the features, and optionally the clips of an implantable device according to any of claims 7 to 9.

11. The device of any preceding claim, wherein the support structure comprises a spring portion that includes one or more flexible loops between the array and the electrical leads that are constructed and arranged so as to urge the electrode array against the spinal cord.

12. The device of any preceding claim, further comprising a dural cuff portion attached to the connecting member and configured to be joined with the dura at or near an access site during implantation of the device into the subject, thereby closing the access site.

13. A method of securing the device according to any of claims 1 to 10 to a vertebra so that the electrode array is in contact with the spinal cord of a subject in need thereof, comprising:
(a) creating an incision in the dura over the dorsal aspect of the spinal canal of the subject;
(b) positioning the arrayed electrodes over the dorsal spinal cord at a location that is essentially symmetrical between the left and right dorsal root entry zones;
(c) lowering the support structure towards the spinal cord so as to compress a spring portion of the deformable support structure and engage the electrodes with the spinal cord within a desired pressure range;
(d) closing the incision around the connecting members; and
(e) securing the support structure to a vertebra of the subject.

14. The method of claim 12, wherein the strap comprises a collar that is structured and arranged to conform to and grasp the lead bundle, and step (e) comprises engaging in the collar a bundle of electrical leads emanating from the device at or near the position that the electrical leads exit the dura.

15. A method for stimulating a spinal cord in a subject in need thereof, comprising delivering an electrical stimulus to a targeted region of the spinal cord by way of a device according to any of claims 1 to 12.

16. A method for stimulating a spinal cord in a subject in need thereof, comprising:
(a) implanting a device according to any of claims 1 to 12 into the spinal cord of the subject; and then
(b) delivering an electrical stimulus to the spinal cord by way of the implanted device.

17. The method of claim 16, wherein the electrical stimulus comprises a pattern of electrical pulses or signals.

18. The method of claim 16, wherein the stimulus is applied so as to inhibit sensation of pain by the subject.

19. The method of claim 16, wherein the stimulus is applied so as to inhibit symptoms of Parkinson's disease, spinal cord injury, or congestive heart failure in the subject.

20. An implantable device according to any of claims 1 to 12 for use n treating back or leg pain, Parkinson's disease, spinal cord injury, or congestive heart failure.

21. A surgical tool configured for securing and positioning an implantable device according to any of claims 1 to 12 for implantation into a subject, the tool comprising:
(a) a central arm;
(b) a coupling joined to the arm and configured for securing the tool to a holding apparatus;
(c) upper and lower members projecting from the arm, adjacent and substantially parallel to each other and spaced apart by a defined span;
(d) a notch on an extremity of each of the upper and lower members, configured to conform to a lead or lead bundle projecting from the implantable device; and
(e) an attachment means on each of the upper and lower members, each configured to receive suture binding the implantable device to the respective member.

22. The tool of claim 21, wherein the span is defined such that when the support structure of the implantable device is positioned beneath and adjacent to the lower member, the device may be secured to a strap passing through the span so as to bridge the lamina of a vertebrae of the patient such that the array of electrodes is urged against the subject's spinal cord.

23. The tool of claim 21 or claim 22, wherein the span is about 0.5 to 0 cm.

24. The tool of any of claims 21 to 23, wherein the attachment on each of the upper and lower members is a hole in the respective member between the extremity and the arm.

25. The tool of any of claims 21 to 24, further comprising a plurality of holes arranged up the arm so that a suture securing the implantable device to the projecting members can pass through the holes, allowing the device to be released by severing the suture near the arm above the projecting members.

26. A surgical tool according to any of claims 21 to 25, in combination with an implantable device according to any of claims 1 to 12.

27. A method of implanting an electrode array onto the spinal cord of a patient in need thereof, comprising:
(a) securing the electrode array to a surgical tool according to any of claims 21 to 25;
(b) positioning the tool so that the electrode array contacts the spinal cord of the patient;
(c) surgically securing the electrode array to one or more anatomical structures of the patient; and
(d) releasing the electrode array from the surgical tool.

28. The method of claim 27, which is a method of implanting an implantable device according to any of claims 1 to 12.

29. The method of claims 27 or claim 28, wherein step (a) comprises securing the electrode array to the surgical tool by way of suture.

30. A surgical tool according to any of claims 21 to 25 for use in treating back or leg pain, Parkinson's disease, spinal cord injury, or congestive heart failure.
